(19) United States
(12) Patent Application Publication
(10) Pub. No.: US 2006/0032093 A1
(43) Pub. Date: Feb. 16, 2006

(54) Orthopedic shoe

(76) Inventor: Antonietta Vannini, Bologna (IT)

Correspondence Address: MODIANO & ASSOCIATI, Via Meravigli, 16, MILAN, 20123, IT

(21) Appl. No.: 11/196,337

(22) Filed: Aug. 4, 2005

(30) Foreign Application Priority Data
Aug. 13, 2004 (IT) BO2004A000527

(51) Int. Cl. A43B 7/20; A61F 5/14; A43B 7/24

(52) U.S. Cl. 36/142; 36/89; 36/140

(57) ABSTRACT

An orthopedic shoe of the type suitable to correct supinate talipes equinovarus, constituted by an upper and a base and substantially shaped like a boot, its particularity consisting in that proximate to one of its portions, arranged at the ankle, it is inclined at a predefined angle with respect to the axial plane of the leg and in a pronating direction for the foot, so as to hypercorrect its pathological supination.

### BACKGROUND OF THE INVENTION

In individuals affected by supinate talipes equinovarus, a neurogenic dysmorphism caused by central lesions at the cerebral level, the foot is arranged so that the sole is flexed and rests on the ground only with the forefoot (talipes equinus) and is rotated about its longitudinal axis, so that the sole is directed inward and rests only on the outer side (supination); further, talipes equinus induces knee recurvation (genu recurvatum).

The consequent motor difficulties, the incorrect posture of the foot and the limited resting surface expose patients affected by this disorder to falls; in particular, in hemiplegic individuals affected by modest spastic palsy, the malposition of the foot worsens at each attempt to flex the paretic limb, and indeed the hemiplegic individual is forced to walk with the limb fixed in the extended position and by sliding with his foot against the surface of the floor in order to avoid spasm during supinating torsion. Moreover, by resting only the outer edge of the forefoot, the patient constantly runs the risk of spraining the tibiotarsal joint, which is further characterized by lack of flexibility.

In order to contain the foot in the functional position, it is known to use various ortheses, such as peroneal springs, Codivilla springs, and others, which are inserted in ordinary shoes and are fixed to the leg of the patient by means of appropriately provided closure systems, for example of the Velcro type.

However, these ortheses may shift within the shoe during walking, becoming uncomfortable and losing their effectiveness, and in any case only partially improve the gait style and confidence, reducing supination of the foot by at most 20-40%.

In order to correct and support the paretic limb better, it is known to resort to custom-made orthopedic shoes, the manufacture of which requires taking a cast of the limb of the patient, obtaining a model thereof and shaping the shoe thereon.

In order to improve the posture of the foot, these shoes can have appropriately arranged rigid inserts and/or couplings for the insertion of ortheses.

However, by having to first provide a negative cast and the corresponding model of the limb of each individual patient, the cost of these custom-made shoes is consequently very high; further, such shoes in any case do not allow to control the foot entirely, as regards both the talipes equinus and the supination that affect it.

This occurs because the cast, by being taken on the paretic limb, duplicates its defects; further during the taking of said cast the patient may unintentionally keep the limb malpositioned or said limb may be subjected temporarily to a physical variation (swelling or the like), affecting the manufacture of the shoe.

Therefore, said custom-made shoes do not allow optimum correction of the pathological dynamics of the foot and not allow to contrast the recurvation of the knee induced by talipes equinus.

### SUMMARY OF THE INVENTION

The aim of the present invention is to solve the problems noted above, by providing an orthopedic shoe which is capable of completely controlling the foot as regards its talipes equinus and supination, correcting or preventing its dysmorphism.

Within this aim, an object of the invention is to provide an orthopedic shoe which avoids the onset of induced recurvation of the knee.

Another object of the invention is to provide an orthopedic shoe which has statistically determined correction criteria, i.e., can be manufactured without the aid of individual casts of the limb of each patient.

Another object of the invention is to provide an orthopedic shoe which, by way of its particular constructive characteristics, is capable of giving the greatest assurances of reliability and safety in use.

Another object of the present invention is to provide an orthopedic shoe that is simple, relatively easy to provide in practice, effective in operation, and further competitive from an economic standpoint.

This aim and these and other objects that will become better apparent hereinafter are achieved by an orthopedic shoe of the boot type suitable to correct supinate talipes equinovarus, constituted by an upper and a base, characterized in that proximate to one of its portions, arrangeable at the ankle of a user, it is inclined at a predefined angle with respect to the axial plane of the leg and in a pronating direction for the foot, so as to hypercorrect its pathological supination.

### BRIEF DESCRIPTION OF THE DRAWINGS

Further characteristics and advantages of the invention will become better apparent from the following detailed description of a preferred but not exclusive embodiment of an orthopedic shoe according to the invention, illustrated by way of non-limiting example in the accompanying drawings, wherein:

FIG. 1 is a perspective view of an orthopedic shoe according to the invention;

FIG. 2 is a side view of an orthopedic shoe;

FIG. 3 is another side view of an orthopedic shoe;

FIG. 4 is a rear view of an orthopedic shoe according to the invention.

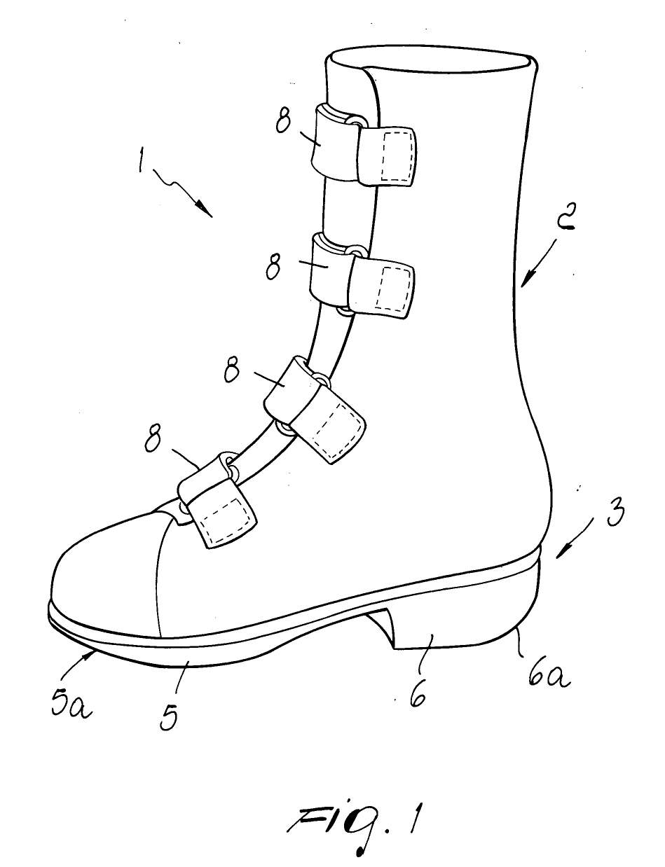

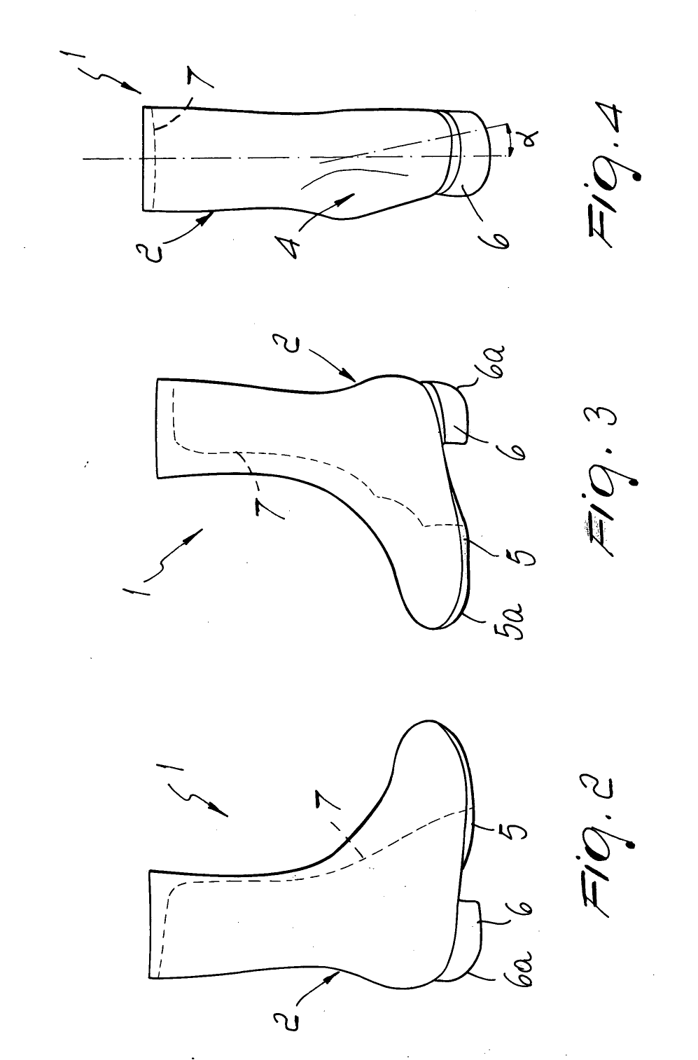

### DESCRIPTION OF THE PREFERRED EMBODIMENTS

With reference to the figures, the reference numeral 1 generally designates an orthopedic shoe according to the invention.

The shoe 1, which is substantially shaped like a boot, is constituted by an upper 2 and a base 3, whose shape is determined according to corrective criteria which are determined statistically on the basis of accurate measurements taken on a sample of hemiplegic patients affected by supinate talipes equinovarus; in this manner, the shoe 1 does not require, in order to be manufactured, a cast of the limb of each patient, but is instead mass-produced.

In particular, the studies that have been conducted have led to the creation of a shoe 1 which, proximate to a portion 4 arranged in use at the ankle of a user, is inclined by a predefined angle a, comprised between 15° and 35° and in particular substantially equal to 30°, with respect to the axial plane of the leg and in the pronating direction for the foot, so as to hypercorrect its pathological supination.

Obviously, the base 3 is shaped suitably so as to place the entire sole of the foot in contact with the ground.

The shoe 1 further has a thick sole 5 provided with a heel 6 for raising the heel of the foot by at least 20 mm with respect to the metatarsal support portion, so as to induce in the patient a bending of the knee which contrasts the recurvation thereof induced by talipes equinus.

The sole 5 is provided with a front bevel 5a and the heel 6 is provided with a rear bevel 6a, which improve the rolling of the shoe 1 during support and therefore allow greater stability of the patient during walking, thus compensating for the mentioned lack of flexibility of the tibiotarsal joint.

The shoe 1 comprises a rigid insert 7, which is arranged laterally and to the rear in the upper 2 at the tarsal, metatarsal, tibial and peroneal portions, and is rigidly coupled to the base 3, so as to ensure the functional positioning of the foot rotated in corrective pronation.

Fastening of the shoe 1 is ensured by a plurality of straps 8 with Velcro closure, which allow the patient to provide optimum adjustment of the fastening force in order to keep the foot in a functional position.

In practice it has been found that the invention fully achieves the intended aim and objects, since the shoe 1, by being rotated at the angle a at the height of the portion 4 and being provided with the rigid insert 7, forces the foot of the patient to perform a pronating rotation which corrects supination, controlling its inversion instability during support; conveniently, the heel 6 of the sole 5 allows the patient to flex the knee, avoiding and/or correcting its recurvation induced by talipes equinus.

Further, as shown, the front bevel 5a of the sole 5 and the rear bevel 6a of the heel 6 improve the rolling of the shoe 1 and consequently also improve the walking of the hemiplegic patient.

Advantageously, the shoe 1 is not custom-made but mass-produced, with a consequent reduction of production costs.

Further benefits from an economic standpoint also arise from the fact that the shoe 1, by allowing more stable and safer walking, allows the patient to avoid the need for expensive aids such as sticks or walkers.

Conveniently, in individuals undergoing rehabilitation therapies, the use of the shoe 1 allows earlier recovery of the ability to walk, with evident benefits.

The invention thus conceived is susceptible of numerous modifications and variations, all of which are within the scope of the appended claims; all the details may be further replaced with other technically equivalent ones.

In the embodiments described, individual characteristics, given in relation to specific examples, may actually be interchanged with other different characteristics that exist in other embodiments.

Moreover, it is noted that anything found to be already known during the patenting process is understood not to be claimed and to be the subject of a disclaimer.

The embodiment of the present invention shall be carried out in the most scrupulous compliance with the statutory and regulatory provisions related to the products of the invention or correlated thereto and following any required authorization of the corresponding competent authorities, with particular reference to regulations related to safety, environmental pollution and health.

In practice, the materials used, as well as the shapes and the dimensions, may be any according to requirements and to the state of the art, without thereby abandoning the scope of the protection of the appended claims.

The disclosures in Italian Patent Application No. BO2004A000527 from which this application claims priority are incorporated herein by reference.

What is claimed is:

1. An orthopedic shoe of a boot type for correcting supinate talipes equinovarus, comprising an upper and a base, and wherein said upper comprises a portion, arranged in use at the ankle of a user and which is shaped with an inclination of a predefined angle with respect to an axial plane of the leg of the user and in a pronating direction, so as to hypercorrect a pathological supination.

2. The orthopedic shoe of claim 1, wherein said predefined angle is comprised between 15° and 35°.

3. The orthopedic shoe of claim 1, comprising a metatarsal support portion and a thick sole provided with a heel with a thickness suitable for raising the heel of the foot of a user by at least 20 mm with respect to said metatarsal support portion.

4. The orthopedic shoe of claim 3, wherein said sole has a front bevel and said heel has a rear bevel, said front and rear bevels, for increasing rolling of the shoe in order to improve walking.

5. The orthopedic shoe of claim 4, further comprising a rigid insert, which is arranged inside said upper and is rigidly connected to said base for functional positioning of the foot of a user.

6. The orthopedic shoe of claim 5, wherein said rigid insert is arranged in said upper so as to be located, in use, substantially laterally and to a rear part of the upper, at least at a tarsal, metatarsal, tibial and peroneal portions of the limb of a user.
